(19) United States
(12) Patent Application Publication
(10) Pub. No.: US 2026/0133206 A1
(43) Pub. Date: May 14, 2026

(54) MISFOLDED PROTEIN DETECTION IN THE EYE

(71) Applicant: LACRISTAT, LLC, San Jose, CA (US)

(72) Inventor: Dimitri Azar, Chicago, IL (US)

(21) Appl. No.: 19/389,501

(22) Filed: Nov. 14, 2025

Related U.S. Application Data
(60) Provisional application No. 63/720,723, filed on Nov. 14, 2024.

(51) Int. Cl. G01N 33/68

(52) U.S. Cl. CPC G01N 33/6896; G01N 2800/2821; G01N 2800/2835

(57) ABSTRACT

A method for determining the presence of soluble, misfolded protein in or on the eye of a subject is described. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the presence of soluble, misfolded protein in the eye. Use of the method for diagnosing disease associated with protein misfolding in a subject is also described.

### CROSS REFERENCE TO RELATED APPLICATIONS

This application claims priority to U.S. Provisional Application No. 63/720,723, filed on Nov. 14, 2024, which is incorporated herein by reference.

### BACKGROUND

The accumulation and deposition of α-synuclein aggregates in brain tissue is the main event in the pathogenesis of different neurodegenerative disorders referred to as synucleinopathies. These include Parkinson's disease, dementia with Lewy bodies and multiple system atrophy. Diagnosis of these disorders mainly relies on the recognition of clinical symptoms, which unfortunately typically provides a diagnosis when the neurodegeneration is already in an advanced phase.

The seed amplification assay (SAA) has been developed for identifying misfolded proteins. See Concha et al., Nat. Protoc., 18(4):1179-1196 (2023). In these assays, α-synuclein seeds circulating in biological fluids are amplified by a cyclical process that includes aggregate fragmentation into smaller self-propagating seeds, followed by elongation at the expense of recombinant α-synuclein. Amplification of the seeds allows detection by fluorescent dyes specific for amyloids, such as thioflavin T. However, such assays require obtaining a sample from a subject, and tedious amplification procedures. Accordingly, there remains a need for a more convenient method of determining the presence of misfolded proteins such as α-synuclein in a subject.

### SUMMARY OF THE INVENTION

In one aspect, the present invention provides a method for determining the presence of soluble, misfolded protein in or on the eye of a subject. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the presence of soluble, misfolded protein in the eye.

In some embodiments, the protein is a neural protein. In further embodiments, the neural protein is α-synuclein, β-amyloid, or tau protein, and in yet further embodiments the neural protein is α-synuclein.

In some embodiments, the step of determining if a detectable amount of misfolded and/or aggregated protein comprises contacting the incubation mixture with a misfolding indicator. In further embodiments, the protein aggregation or misfolding indicator is Thioflavin T. In yet further embodiments, the subject is a human subject. In additional embodiments, the incubation and determining if a detectable amount of misfolded and/or aggregated protein is present are conducted ex vivo.

In some embodiments, the presence of soluble, misfolded protein is determined in the tear fluid on the eye. In other embodiments, the presence of soluble, misfolded protein is determined in the aqueous humor and/or the vitreous humor of the eye.

Another aspect of the invention provides a method of diagnosing disease associated with protein aggregation in a subject. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the subject has a disease associated with protein aggregation.

In some embodiments, the protein is a neural protein. In further embodiments, the neural protein is α-synuclein, β-amyloid, or tau protein, and in yet further embodiments the neural protein is α-synuclein. In some embodiments where the neural protein is α-synuclein the disease associated with protein aggregation is Parkinson's disease. In additional embodiments, the method further comprising treating the subject diagnosed as having a disease associated with protein aggregation.

In some embodiments, the step of determining if a detectable amount of misfolded and/or aggregated neural protein comprises contacting the incubation mixture with a protein aggregation or misfolding indicator. In further embodiments, the protein aggregation or misfolding indicator is Thioflavin T. In some embodiments, the subject is a human subject. In additional embodiments, the incubation and determining if a detectable amount of misfolded and/or aggregated protein is present are conducted ex vivo.

In some embodiments, the presence of soluble, misfolded protein is determined in the tear fluid on the eye. In other embodiments, the presence of soluble, misfolded protein is determined in the aqueous humor and/or the vitreous humor of the eye.

### DETAILED DESCRIPTION OF THE INVENTION

The present invention relates a method for determining the presence of soluble, misfolded protein in or on the eye of a subject. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the presence of soluble, misfolded protein in or on the eye. Use of the method for diagnosing disease associated with protein aggregation in a subject is also provided.

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously. In the case of a prognosis for Parkinson's Disease, the prognosis categorizes the relative severity of the Parkinson's Disease, which can be used to guide selection of appropriate therapy.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

As used herein, the term “about” in conjunction with a number is intended to include ±10% of the number. In other words, “about 10” may mean from 9 to 11.

As used herein and in the appended claims, the singular forms “a”, “and”, and “the” include plural referents unless the context clearly dictates otherwise. Thus, for example, reference to “a sample” also includes a plurality of such samples and reference to “the α-synuclein protein” includes reference to one or more protein molecules, and so forth.

### Determining the Presence of Soluble, Misfolded Protein

In one aspect, the present invention provides a method for determining the presence of soluble, misfolded protein in or on the eye of a subject. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the presence of soluble, misfolded protein in the eye. In some embodiments, determining the presence includes detecting whether soluble, misfolded protein is present in or on the eye or not, while in other embodiments determining the presence includes determining the amount of soluble, misfolded protein in or on the eye.

In some embodiments, the protein is a neural protein. Neural proteins are proteins specific to nerve cells that are crucial for their structure, function, and communication. In some embodiments, the neural proteins are capable of misfolding, aggregation, and/or are associated with neurodegeneration. In further embodiments, the neural protein is a non-synaptic protein such as α-synuclein, β-amyloid, or tau protein.

As used herein, “monomeric neural protein” (e.g., α-synuclein protein) refers to single α-synuclein protein molecules in their native, nonpathogenic, folded configuration. “Soluble, misfolded neural protein” refers to misfolded monomers or aggregates of a neural protein that remain in solution. Examples of soluble, misfolded neural protein may include any number of aggregated misfolded neural protein monomers so long as the misfolded neural protein remains soluble. For example, soluble, misfolded α-synuclein protein may include aggregates of between 2 and about 50 units of misfolded α-synuclein protein monomer. In some examples, aggregates may be referred to as oligomers or polymers. In some examples, aggregation may be referred to as oligomerization or polymerization.

Soluble, misfolded neural protein (e.g., α-synuclein protein) may aggregate or oligomerize to form insoluble aggregates and/or higher oligomers, leading to neural protein aggregates in the form of protofibrils, fibrils, and eventually plaques or inclusion bodies. “Seeds” or “nuclei” refer to misfolded neural protein or short fragmented fibrils, particularly soluble, misfolded neural protein, with catalytic activity for inducing further misfolding, oligomerization, and/or aggregation. Such nucleation-dependent polymerization may be characterized by a slow lag phase wherein aggregated nuclei may form, which may then catalyze rapid formation of further and/or larger aggregates. The lag phase may be minimized or removed by addition of pre-formed nuclei or seeds. In some examples, “seeds” or “nuclei” may exclude un-aggregated monomers of neural protein.

As used herein, “soluble” species, including soluble misfolded neural protein (e.g., α-synuclein), may form a solution in biological fluids under physiological conditions, whereas “insoluble” species may be present as precipitates, fibrils, deposits, tangles, or other non-dissolved forms in such biological fluids. Examples of insoluble species include fibrils of Aβ, αS, tau, and the like. A species that dissolves in a non-biological fluid but not a biological fluid under physiological conditions may be considered insoluble. For example, fibrils of α-synuclein and the like may be dissolved in a solution of, e.g., a surfactant such as sodium dodecyl sulfate (SDS) in water, but may still be insoluble in one or more of the mentioned biological fluids under physiological conditions, and are therefore considered insoluble herein.

As used herein, a “misfolded neural protein” is a protein that no longer contains all or part of the structural conformation of the protein as it exists when involved in its typical, nonpathogenic normal function within a biological system. A misfolded protein may aggregate. A misfolded protein may localize in protein aggregate. A misfolded protein may be a non-functional protein. A misfolded protein may be a pathogenic conformer of the protein. Monomeric α-synuclein protein compositions may be provided in native, nonpathogenic confirmations without the catalytic activity for misfolding, oligomerization, and aggregation associated with seeds. Monomeric neural protein compositions may be provided in seed-free form. While the aggregation mechanism of α-synuclein is still being investigated, there is evidence of a structured intermediate rich in beta structure that can be the precursor of aggregation and, ultimately, Lewy bodies. Kim et al., Chembiochem. 8 (14): 1671-4 (2007).

The method includes the step of incubating the incubation mixture to form a misfolded protein aggregate from the monomeric protein. The incubation can take place in the eye (i.e., in vivo), or alternately the incubation and determining if a detectable amount of misfolded and/or aggregated protein is present can be conducted ex vivo. The incubation mixture is incubated for a sufficient period of time to form a misfolded neural protein aggregate from the monomeric, folded neural protein. In some embodiments, incubation can be carried out for a period ranging from 0.5 hours to 24 hours. In further embodiments, the incubation can be carried out for a period ranging from 1 to 6 hours.

When conducted ex vivo, the incubation may be conducted at a temperature between about 35° C. and about 40° C. In some embodiments, ex vivo incubation can include a cycle of both incubation and de-aggregation, with an incubation cycle lasting from 0.3 to 1 hour. De-aggregating the incubation mixture may include one or more types of physical disruption selected from: sonication, stirring, shaking, freezing/thawing, laser irradiation, autoclave incubation, high pressure, homogenization, and the like. The incubation mixture can include various different buffer compositions. The buffer composition is effective to maintain the pH of the incubation mixture in a range from about pH 5 to about pH 9, from about pH 6 to about pH 8, from about pH 7 to about pH 8, or at a pH of about 7. In some embodiments, the incubation mixture comprises one or more of the buffers Tris-HCL, MES, PIPES, MOPS, BES, TES, and HEPES.

The incubation mixture can include various different concentrations of the monomeric neural protein (e.g., α-synuclein protein). In some embodiments, the incubation mixture may include the monomeric α-synuclein protein in a concentration, or in a concentration range, of one or more of: between about 500 nM and about 200 μM; between about 1 μM and about 100 μM; between about 5 μM to about 50 μM.; or between about 10 μM and about 40 μM.

As used herein, aggregates of neural protein (e.g., α-synuclein protein) refer to non-covalent associations of protein including soluble, misfolded neural protein. Aggregates of neural proteins may be “de-aggregated”, broken up, or disrupted to release smaller fragments or aggregates, e.g., soluble, misfolded neural protein and fragmented fibrils. The catalytic activity of a collection of misfolded neural protein aggregate seeds may scale, at least in part with the number of seeds in a mixture.

In some embodiments, the neural protein is α-synuclein. As used herein, “αS” or “α-synuclein” refers to full-length, 140 amino acid α-synuclein protein, i.e., “αS-140.” Other isoforms or fragments may include “αS-126,” alpha-synuclein-126, which lacks residues 41-54, e.g., due to loss of exon 3; and “αS-112” alpha-synuclein-112, which lacks residue 103-130, e.g., due to loss of exon 5. Various αS isoforms may include and are not limited to αS-140, αS-126, and αS-112. Various α-synuclein peptides may be associated with neuronal damage associated with synucleinopathy such as Parkinson's disease.

In some embodiments, the neural protein is β-amyloid protein (Aβ). Aβ is a cleavage product of the amyloid precursor protein, and includes peptides of 36-43 amino acids that are the main component of amyloid plaque. In Alzheimer's disease, Aβ peptides accumulate into plaques between neurons. Aβ molecules can aggregate to form flexible soluble oligomers which may exist in several forms. Various Aβ isoforms may include and are not limited to Aβ₄₀ and Aβ₄₂. Olsson et al., J Biol Chem., 289(3): 1540-1550 (2014). Certain misfolded oligomers (known as “seeds”) can induce other Aβ molecules to also take the misfolded oligomeric form, leading to a chain reaction akin to a prion infection. The buildup of Aβ is also thought to initiate the abnormal tau aggregation process.

In some embodiments, the neural protein is Tau (“tubulin associated unit”) protein. Tau proteins form a group of six highly soluble protein isoforms produced by alternative splicing from the gene MAPT (microtubule-associated protein tau). In healthy neurons, tau stabilizes microtubules, which are essential for transport within the cell. Pathologies and dementias of the nervous system such as Alzheimer's disease and Parkinson's disease are associated with tau proteins that have become hyperphosphorylated insoluble aggregates called neurofibrillary tangles.

### Assay in or on the Eye

The invention provides a method for determining the presence of soluble, misfolded protein in or on the eye of a subject. The method includes contacting the eye of the subject with a monomeric protein to form an incubation mixture. In some embodiments, the presence of soluble, misfolded protein is determined in the tear fluid on the eye, while in other embodiments, the presence of soluble, misfolded protein is determined in the aqueous humor and/or the vitreous humor of the eye.

In or on the eye refers to carrying the method out on the surface of the eye (i.e., in the tear fluid) and/or carrying out the method in a liquid within the eye, such as the aqueous humor or the vitreous humor. The tear fluid of the eye can easily be contacted with monomeric protein and aggregation or misfolding indicator using (for example) an eye dropper or pipette, while liquid within the eye can be contacted using a syringe. See for example Muniyandi et al., Invest Ophthalmol Vis Sci., 65(1):18 (2024).

The method includes determining if a detectable amount of misfolded and/or aggregated protein (e.g., α-synuclein) is present in or on the eye. Detection of misfolded and/or aggregated protein indicates the presence of soluble, misfolded protein in or on the eye.

In some embodiments, the method includes the step of contacting the incubation mixture with a protein aggregation or misfolding indicator to determining if a detectable amount of misfolded and/or aggregated protein is present in the incubation mixture. The incubation mixture is formed in the eye when a monomeric protein is introduced into or on the eye. The protein aggregation or misfolding indicator can be characterized by exhibiting an indicating state in the presence of misfolded protein and a non-indicating state in the absence of misfolded protein. Determining the presence of the soluble, misfolded protein in or on the eye may include detecting the indicating state of the indicator of misfolded protein. The indicating state of the indicator and the non-indicating state of the indicator may be characterized by a difference in fluorescence, light absorption or radioactivity depending on the specific indicator. The step of determining the presence of the soluble, misfolded protein in or on the eye may include detecting the difference in fluorescence, light absorption or radioactivity depending on the specific indicator being used. Additional methods that can be used to detect protein aggregate include birefringence and interferometry.

In some embodiments, the protein aggregation or misfolding indicator may include one or more of: Thioflavin T, Congo Red, m-I-Stilbene, Chrysamine G, PIB, BF-227, X-34, TZDM, FDDNP, MeO-X-04, IMPY, NIAD-4, luminescent conjugated polythiophenes, a fusion with a fluorescent protein such as green fluorescent protein and yellow fluorescent protein, derivatives thereof, and the like. A preferred protein aggregation or misfolding indicator is Thioflavin T. When a fluorescent indicator such as Thioflavin T is used, fluorescence can be detected within the eye using conventional means.

In some embodiments, the method may include providing the monomeric, folded protein in labeled form. The monomeric, folded protein in labeled form may include one or more of: a covalently incorporated radioactive amino acid, a covalently incorporated, isotopically labeled amino acid, a covalently incorporated fluorophore, and the like. Detection of the soluble, misfolded protein may include detecting the monomeric, folded protein in labeled form as incorporated into the amplified portion of misfolded protein.

In some embodiments, determining the presence of the soluble, misfolded protein in or on the eye includes determining the amount of the soluble, misfolded protein in the in or on the eye. The amount of the soluble, misfolded protein in the sample may be determined compared to a control sample. The amount of the soluble, misfolded protein in or on the eye may be detected with a sensitivity of at least about one or more of: 80%, 81%, 82%, 83%, 84%, 85%, 86%, 87%, 88%, 89%, 90%, 91%, 92%, 93%, 94%, 95%, 96%, 97%, 98%, 99%, and 100%. The amount of the soluble, misfolded protein detected in or on the eye may be less than about one or more of: 100 nmol, 10 nmol, 1 nmol, 100 pmol, 10 pmol, 1 pmol, 100 fmol, 10 fmol, 3 fmol, 1 fmol, 100 attomol, 10 attomol, and 1 attomol. The amount of the soluble, misfolded protein in or on the eye may be detected in a molar ratio to monomeric, folded protein delivered to the eye. The molar ratio may be less than about one or more of 1:100, 1:10,000, 1:100,000, and 1:1,000,000.

In some embodiments, the levels of soluble, misfolded may be compared to the level of corresponding internal standards in the sample or samples when carrying out the analysis to quantify the amount of soluble, misfolded being detected. Once the presence and/or level of the soluble, misfolded protein has been determined, it can be displayed in a variety of ways. For example, the levels can be displayed graphically on a display as numeric values or proportional bars (i.e., a bar graph) or any other display method known to those skilled in the art. The graphic display can provide a visual representation of the amount of the variance in the biological sample being evaluated.

### Subjects

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Analysis of biological samples from human subjects is of particular interest.

In some embodiments, the subject may be at risk of developing a disease associated with protein misfolding such as Parkinson's disease (PD), of having PD, or being under treatment for PD; at risk of having a disease associated with dysregulation, misfolding, aggregation or disposition of α-synuclein; such as Multiple System Atrophy; having a disease associated with dysregulation, misfolding, aggregation or disposition of α-synuclein; under treatment for a disease associated with dysregulation, misfolding, aggregation or disposition of α-synuclein; and the like. For a description of the risk factors associated with Parkinson's disease, see Ascherio A, Schwartzschild M A, Lancet Neurol., 15(12):1257-1272 (2016).

### Methods of Diagnosing Disease Associated With Protein Misfolding

Another aspect of the invention provides a method of diagnosing disease associated with protein misfolding in a subject. The method includes contacting the eye of the subject with a monomeric protein to form an incubation mixture; incubating the incubation mixture to form a misfolded protein aggregate from the monomeric protein; and determining if a detectable amount of misfolded and/or aggregated protein is present in or on the eye; wherein detection of misfolded and/or aggregated protein indicates the subject has a disease associated with protein misfolding. The method may include determining or diagnosing the presence of a disease associated with protein misfolding in the subject by comparing the amount of the soluble, misfolded protein in or on the eye to a predetermined threshold amount, wherein a level higher than the threshold amount provides a diagnosis of a disease associated with protein misfolding in the subject.

In some embodiments, the protein is a neural protein. In further embodiments, the neural protein is α-synuclein, β-amyloid, or tau protein, and in yet further embodiments the neural protein is α-synuclein.

Protein misfolding disorders (PMDs) include Alzheimer's disease, Parkinson's disease, type 2 diabetes, Huntington's disease, amyotrophic lateral sclerosis, systemic amyloidosis, prion diseases, and the like. PMDs also include disease associated with α-synuclein aggregation. Misfolded aggregates of different proteins may be formed and accumulate. The misfolded aggregates may induce cellular dysfunction and tissue damage, among other effects. In some embodiments, the disease associated with α-synuclein aggregation is Parkinson's disease, while in further embodiments the disease associated with α-synuclein aggregation is Lewy Body Dementia.

The method may include diagnosing Parkinson's Disease (PD) in the subject based on detecting soluble, misfolded α-synuclein protein in or on the eye of the subject. Alphα-synuclein misfolding and aggregation has been shown to be associated with PD pathogenesis. Sahay et al., Curr Protein Pept Sci., 18(7):656-676 (2017). A diagnosis of PD may also include comparing the amount of soluble, misfolded α-synuclein protein in or on the eye of the subject to a control sample taken from a control subject to determine the level of soluble, misfolded α-synuclein protein relative to that present in a healthy subject. The method may include determining or diagnosing the presence of a disease associated with protein misfolding in the subject according to the presence of the soluble, misfolded protein in or on the eye of the subject. The method may include determining or diagnosing the presence of Multiple System Atrophy in the subject according to the presence of the soluble, misfolded α-synuclein protein in or on the eye of the subject.

In some embodiments, the method may be conducted in a subject exhibiting no clinical signs of Parkinson's disease (PD). In other embodiments, the method may be conducted in a subject exhibiting clinical signs of PD. The most recognizable symptom of PD is motor-related dysfunction. However, additional symptoms include autonomic dysfunction, neuropsychiatric problems (mood, cognition, behavior or thought alterations), sensory dysfunction (especially altered sense of smell) and sleep difficulties.

In some embodiments, the method includes treating a subject diagnosed as having a disease associated with protein (e.g., α-synuclein) aggregation with protein (e.g., α-synuclein) modulating therapy. Several novel therapeutics that are targeting α-synuclein homeostasis through various mechanisms are currently under development. The α-synuclein modulating therapy may include inhibiting the production of α-synuclein inhibiting the aggregation of α-synuclein, e.g., with a suitable inhibitor, active or passive immunotherapy approaches, and the like. Therapeutic approaches targeting α-synuclein homeostasis may include active immunization, such as PD01A+ or PD03A+, or passive immunization such as PRX002. The method described herein for detecting the presence of soluble, misfolded α-synuclein can be employed to determine which patients may be treated with an α-synuclein modulating therapy. While there is currently no cure for Parkinson's disease, a variety of drugs are useful for treating the motor symptoms of PD, such as levodopa, dopamine agonists, and monoamine oxidase B inhibitors.

The method may include diagnosing Alzheimer's disease in a subject based on detecting soluble, misfolded Aβ protein in the biological sample. Aβ protein misfolding and aggregation has been shown to be associated with diseases associated with Aβ aggregation such as Alzheimer's disease. Iadanza et al., Nat Rev Mol Cell Biol., 19(12):755-773 (2018). A diagnosis of Alzheimer's disease may also include comparing the amount of soluble, misfolded Aβ protein in the biological sample to a control sample taken from a control subject to determine the level of soluble, misfolded Aβ protein relative to that present in a healthy subject. The method may include determining or diagnosing the presence of a disease associated with A protein aggregation in the subject according to the presence of the soluble, misfolded Aβ protein in the biological sample.

In some embodiments, the sample may be taken from a subject exhibiting no clinical signs of Alzheimer's disease. In other embodiments, the biological sample may be taken from a subject exhibiting clinical signs of Alzheimer's disease. The most recognizable symptom of Alzheimer's disease is cognitive dysfunction, which can be exhibited as short-term memory loss and language problems. Diagnosis of Alzheimer's disease is usually based on behavioral observations, neuropsychology tests, and imaging using computed tomography or magnetic resonance imaging.

In some embodiments, the method includes treating a subject diagnosed as having a disease associated with Aβ protein aggregation with Aβ protein modulating therapy. Several novel therapeutics that are targeting Aβ protein homeostasis through various mechanisms are currently under development. The Aβ modulating therapy may include administration of one or more of: an inhibitor of BACE1 (β-secretase 1); an inhibitor of γ-secretase; and a modulator of Aβ homeostasis, e.g., an immunotherapeutic modulator of Aβ homeostasis. The Aβ modulating therapy may include administration of one or more of: E2609; MK-8931; LY2886721; AZD3293; semagacestat (LY-450139); avagacestat (BMS-708163); solanezumab; crenezumab; bapineuzumab; BIIB037; CAD106; 8F5; or other antibodies raised against Aβ proteins, e.g., as described by Barghorn et al, J. Neurochem., 95, 834-847 (2005), the entire teachings of which are incorporated herein by reference; ACC-001; V950; Affitrope AD02; and the like. Other examples of compounds useful for treating Alzheimer's disease include the NMDA receptor antagonist memantine and the acetylcholinesterase inhibitor donepezil.

The method may include diagnosing Alzheimer's disease in a subject based on detecting soluble, misfolded Tau protein in the biological sample. Tau protein misfolding and aggregation has been shown to be associated with diseases associated with Tau aggregation such as Alzheimer's disease. Iadanza et al., Nat Rev Mol Cell Biol., 19(12):755-773 (2018). A diagnosis of Alzheimer's disease may also include comparing the amount of soluble, misfolded Tau protein in the biological sample to a control sample taken from a control subject to determine the level of soluble, misfolded Tau protein relative to that present in a healthy subject. The method may include determining or diagnosing the presence of a disease associated with Tau protein aggregation in the subject according to the presence of the soluble, misfolded Tau protein in the biological sample.

In some embodiments, the sample may be taken from a subject exhibiting no clinical signs of Alzheimer's disease. In other embodiments, the biological sample may be taken from a subject exhibiting clinical signs of Alzheimer's disease. The most recognizable symptom of Alzheimer's disease is cognitive dysfunction, which can be exhibited as short-term memory loss and language problems. Diagnosis of Alzheimer's disease is usually based on behavioral observations, neuropsychology tests, and imaging using computed tomography or magnetic resonance imaging.

In some embodiments, the method includes treating a subject diagnosed as having a disease associated with Tau protein aggregation with Tau protein modulating therapy. Several novel therapeutics that are targeting Tau protein homeostasis through various mechanisms are currently under development. The Tau modulating therapy may include administration of one or more of immunotherapy, inhibiting tau phosphorylation, inhibiting tau aggregation, reducing tau expression, modulating tau isoforms, and promoting tau clearance. See for example Dominguez-Meijide et al., Brain Sci., 10(11):858 (2020), the disclosure of which is incorporated herein by reference, which describes pharmacological modulators of tau aggregation and spreading.

In some embodiments, the method is used to monitor treatment of a disease associated with protein misfolding in a subject. The level of misfolded protein in or on the eye of the subject under protein (e.g., α-synuclein, Aβ, or Tau) modulating therapy may be determined at different times over a period of time. The method may include determining or diagnosing the subject is one of: responsive to the neural protein modulating therapy according to a change in the soluble, misfolded neural protein over the period of time, or non-responsive to the α-synuclein modulating therapy according to homeostasis of the soluble, misfolded neural protein over the period of time. The method may include treating the subject determined to be responsive to the protein (e.g., α-synuclein) modulating therapy with the protein (e.g., α-synuclein) modulating therapy.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for determining the presence of soluble, misfolded protein in or on the eye of a subject, comprising:
contacting the eye of the subject with a monomeric protein to form an incubation mixture;
incubating the incubation mixture to form a misfolded protein aggregate from the monomeric protein; and
determining if a detectable amount of misfolded and/or aggregated protein is present in or on the eye; wherein detection of misfolded and/or aggregated protein indicates the presence of soluble, misfolded protein in the eye.

2. The method of claim 1, wherein the protein is a neural protein.

3. The method of claim 2, wherein the neural protein is α-synuclein, β-amyloid, or tau protein.

4. The method of claim 2, wherein the neural protein is α-synuclein.

5. The method of claim 1, wherein the step of determining if a detectable amount of misfolded and/or aggregated protein comprises contacting the incubation mixture with a protein aggregation or misfolding indicator.

6. The method of claim 5, wherein the protein aggregation or misfolding indicator is Thioflavin T.

7. The method of claim 1, wherein birefringence or interferometry are used to determine if a detectable amount of misfolded and/or aggregated protein is present in or on the eye.

8. The method of claim 1, wherein the subject is a human subject.

9. The method of claim 1, wherein the presence of soluble, misfolded protein is determined in the tear fluid on the eye.

10. The method of claim 1, wherein the presence of soluble, misfolded protein is determined in the aqueous humor and/or the vitreous humor of the eye.

11. The method of claim 1, wherein the incubation and determining if a detectable amount of misfolded and/or aggregated protein is present are conducted ex vivo.

12. A method of diagnosing disease associated with protein misfolding in a subject, comprising:
contacting the eye of the subject with a monomeric protein to form an incubation mixture;
incubating the incubation mixture to form a misfolded protein aggregate from the monomeric protein; and
determining if a detectable amount of misfolded and/or aggregated protein is present in or on the eye; wherein detection of misfolded and/or aggregated protein indicates the subject has a disease associated with protein misfolding.

13. The method of claim 12, wherein the protein is a neural protein.

14. The method of claim 13, wherein the neural protein is α-synuclein, β-amyloid, or tau protein.

15. The method of claim 13, wherein the neural protein is α-synuclein.

16. The method of claim 15, wherein the disease associated with protein aggregation is Parkinson's disease.

17. The method of claim 12, wherein the step of determining if a detectable amount of misfolded and/or aggregated neural protein comprises contacting the incubation mixture with a protein aggregation or misfolding indicator.

18. The method of claim 17, wherein the protein aggregation or misfolding indicator is Thioflavin T.

19. The method of claim 12, wherein birefringence or interferometry are used to determine if a detectable amount of misfolded and/or aggregated protein is present in or on the eye.

20. The method of claim 12, wherein the subject is a human subject.

21. The method of claim 12, wherein the presence of soluble, misfolded protein is determined in the tear fluid on the eye.

22. The method of claim 12, wherein the presence of soluble, misfolded protein is determined in the aqueous humor and/or the vitreous humor of the eye.

23. The method of claim 12, wherein the incubation and determining if a detectable amount of misfolded and/or aggregated protein is present are conducted ex vivo.

24. The method of claim 12, further comprising treating the subject diagnosed as having a disease associated with protein misfolding.
